(19) United States
(12) Patent Application Publication
(10) Pub. No.: US 2019/0117933 A1
(43) Pub. Date: Apr. 25, 2019

(54) DEVICES AMD METHODS FOR BRAIN STIMULATION

(71) Applicant: Daniel J. Couser, Glenmoore, PA (US)

(72) Inventor: Daniel J. Couser, Glenmoore, PA (US)

(21) Appl. No.: 16/180,885

(22) Filed: Nov. 5, 2018

Related U.S. Application Data
(63) Continuation-in-part of application No. 15/972,171, filed on May 6, 2018.
(60) Provisional application No. 62/631,869, filed on Feb. 18, 2018.
(60) Provisional application No. 62/501,872, filed on May 5, 2017.

(51) Int. Cl. A61M 21/02; A61H 23/02

(52) U.S. Cl. CPC A61M 21/02; A61M 2021/0022; A61H 23/02

(57) ABSTRACT

A handheld device configured to provide tactile stimulation to a head of a human user. The handheld device comprises an outer housing sized and shaped to fit ergonomically and substantially within a hand of the human user. The outer housing defines an internal space and skin facing surface for placement adjacent to or proximate the user's head. A controller is contained within internal space of the outer housing, wherein the controller is configured to generate a signal corresponding to a pattern sequence. A vibration unit is in electronic communication with the controller, the vibration unit being configured to be actuated in response to a signal from the controller so as to generate vibrations that correspond to the pattern sequence.

### CROSS-REFERENCE TO RELATED APPLICATIONS

This application is a continuation-in-part of U.S. Ser. No. 15/972,171, which claims the benefit of priority under 35 U.S.C. § 119(e) from U.S. Provisional Patent Application Ser. No. 62/501,872, filed on May 5, 2017, and U.S. Provisional Application Ser. No. 62/631,869, filed on Feb. 18, 2018. The present application claims the direct benefit of and priority to U.S. Application Ser. No. 62/631,869, filed on Feb. 18, 2018. The contents of each application in this paragraph are incorporated by reference herein in their entireties.

### FIELD OF THE INVENTION

The present disclosure generally relates to devices and methods for brain stimulation, and in particular to systems and method for anxiety control.

### BACKGROUND

Millions of individuals suffer from clinically diagnosed anxiety on a daily basis. There are countless other people who suffer and experience chronic and intense stress on a frequent basis, if not each day. While there are various medications to treat anxiety and chronic stress, there are no reliable, widely accepted non-pharmaceutical treatment options available to sufferers, that are able to effectively prevent an anxiety attack, or stop an attack while it is happening.

When an anxiety attack occurs, the body typically reacts in an involuntary fashion by instituting a “fight or flight” condition or response. This condition or response is usually accompanied by an often overwhelming sense of fear or dread that generally has no immediate or future threat directly posed to the individual. Health and medical counselors generally advise individuals who experience such attacks to use controlled breathing and other grounding techniques as a means to help control or subdue such an anxiety attack. Unfortunately, it is extremely difficult for an individual experiencing an anxiety attack to subdue the symptoms simply through breathing techniques, especially when the effects of a fight or flight response are involuntarily and acutely manifested with both physical and mental characteristics.

When elevated anxiety or high stress levels commence, it is known that the person's brain emits higher levels of beta waves while also emitting decreased levels of alpha and theta waves. These high anxiety brain wave patterns (elevated and dominant beta waves) are associated with rapid and frenzied thoughts within the brain. It has been postulated that if an effective means existed for controlling higher levels of beta waves while also encouraging higher levels of alpha and theta waves, such a means, device or system could directly and substantively benefit those who experience anxiety attacks and uncontrolled high stress levels.

Several studies have been undertaken and positively show that one way to address the problem of controlling an anxiety attack is through a form of brainwave influencing or brain state induced synchronization. Such studies have shown that a subject's brain tends to follow or adjust to match certain frequencies that are imparted to or emitted to the subject's head or cranium. More particularly, in a 2007 study, cortical stimulation with repetitive frequencies of 1 to 8 Hz was shown to increase phase synchronization in all EEG frequency bands (Will and Berg, 2007). Additionally, a further study evaluated treatment for cortisol induced anxiety in mice through use of rhythmical flickering photic stimulation at alpha frequencies from 9 to 11 Hz. This latter study showed improved performance on various behavioral tasks assessing anxiety, locomotor activity, social interaction, and despair (Kim, et al., 2016).

### SUMMARY OF THE INVENTION

Embodiments of the present disclosure includes a handheld device configured to provide tactile stimulation to a head of a human user. The handheld device comprises an outer housing sized and shaped to fit ergonomically and substantially within a hand of the human user. The outer housing defines an internal space and skin facing surface for placement adjacent to or proximate the user's head. A controller is contained within internal space of the outer housing, wherein the controller is configured to generate a signal corresponding to a pattern sequence. A vibration unit is in electronic communication with the controller, the vibration unit being configured to be actuated in response to a signal from the controller so as to generate vibrations that correspond to the pattern sequence.

Another embodiment of the disclosure is a handheld device configured to provide tactile stimulation to a head of a human user. The handheld device comprises an outer housing sized and shaped to fit ergonomically and substantially within a hand of the human user. The outer housing defines an internal space and skin facing surface for placement adjacent to or proximate the user's head. The handheld device may include a controller contained within internal space of the outer housing. The controller includes a processor and a memory unit. The memory includes stored thereon a data file. The data file includes a pattern sequence. The processor is configured to generate a signal corresponding to the pattern sequence contained within the data file. The handled device includes a vibration unit in electronic communication with the processor of the controller. The vibration unit is configured to be actuated in response to a signal from the processor so as to generate vibrations that correspond to the pattern sequence.

### BRIEF DESCRIPTION OF THE DRAWINGS

For the purposes of illustrating the invention, the attached drawings show certain aspects and embodiments that are presently preferred. However, it should be understood that the invention is not limited to the precise configuration and particular components or system elements as shown in the accompanying drawings, but rather is further disclosed and claimed according to the attached claims. In the drawings:

FIG. 1 is a side view of a device illustrating its internal components according to an embodiment of the present disclosure;

FIG. 2A shows an exemplary pattern sequence in accordance with an embodiment of the present disclosure;

FIG. 2B shows an exemplary pattern sequence in accordance with another embodiment of the present disclosure;

FIG. 2C shows an exemplary a pattern sequence in accordance with another embodiment of the present disclosure;

FIG. 3 illustrates the device placed on a user in accordance with an embodiment of the present disclosure.

FIG. 4A is a perspective view of a handheld device according to an embodiment of the present disclosure;

FIG. 4B is a side view of the handheld device shown in FIG. 4A;

FIG. 4C is another side view of the handheld device shown in FIGS. 4A and 4B;

FIG. 4D is an exploded view of the handheld device shown in FIGS. 4A-4C;

FIG. 4E is a block diagram of for a system providing tactile stimulation in accordance with another embodiment of the present disclosure;

FIG. 4F is a block diagram of a system apparatus for providing tactile stimulation in accordance with another embodiment of the present disclosure.

FIG. 5 illustrates a system for providing stimulation according to another embodiment of the present disclosure;

FIG. 6 illustrates a system for providing stimulation according to another embodiment of the present disclosure;

FIG. 7A is a schematic view of wearable device for providing stimulation according to another embodiment of the present disclosure;

FIG. 7B is another a schematic view of wearable device shown in FIG. 7A;

FIG. 8 is a schematic diagram illustrating another embodiment of the device;

FIG. 9 is a top view of the device configured to the adhere to the user;

FIG. 10 is perspective view of an eye-glass device configured to provide stimulation to the user in accordance with another embodiment of the present disclosure;

FIG. 11 is perspective view of a device configured to be worn over the ear of the user;

FIG. 12 is a schematic view of device configured to worn on a user's finger;

FIG. 13 is a ring device according to an embodiment of the present disclosure;

FIG. 14 is another embodiment of a ring device illustrated in FIG. 13;

FIG. 15 is a perspective view of a device disposed in a cover a smart phone;

FIG. 16 is a vibratory unit used in the device according to another embodiment of the present disclosure;

FIG. 17 is a perspective view of a handheld device according to another embodiment of the present disclosure;

FIG. 18 is an end view of the handheld device shown in FIG. 17;

FIG. 19 is another end view of the handheld device shown in FIG. 18;

FIG. 20 is a side view of a handheld device shown in FIG. 17;

FIG. 21 is another side view of a handheld device shown in FIG. 17;

FIG. 22 is a bottom view of a handheld device shown in FIG. 17;

FIG. 23 is a top view of a handheld device shown in FIG. 17; and

FIG. 24 is a cross-sectional view of the handheld device taken along line 8-8 in FIG. 22.

### DETAILED DESCRIPTION OF EXEMPLARY EMBODIMENTS

The present disclosure seeks to address and resolve the problems experienced by individuals suffering from involuntary anxiety attacks and involuntary elevated stress levels. Embodiments of the present disclosure allow users to discretely control and potentially alleviate involuntary anxiety attacks and high stress states. In particular, embodiments of the present disclosure are configured to induce or influence brainwave synchronization by imparting stored waveform signals adjacent to the user's cranium. Through the induced synchronization of the user's brainwaves or brain state, embodiments of the present disclosure may reduce or eliminate anxiety attacks, prevent stress and achieve desired brainwave states or conditions.

The devices described in the present disclosure generate stimuli that is imparted to users in order to encourage synchronization of the user's brainwaves to mimic the frequency of the desired stimuli. Frequency waves, such as signals with various pattern sequences, may be used as a baseline or reference to which the user's brainwaves are encourage to synchronization or follow. The pattern sequence may be a signal with a pattern that varies over a period of time. Furthermore, a pattern sequence may include a repeating pattern sequence whereby the pattern repeats over time. Depending on one or more frequencies, amplitudes, and pitches of the waves, a range of brainwave states can be achieved, resulting in various beneficial effects. For example, stimuli in the form of one signal response may generate one effect in the brain state while other types of signal responses could generate a different effects or changes in the brain state. Accordingly, embodiments of the present disclosure utilized a wide range of stimuli and signal responses to induce the desired brain states, as will be further described below.

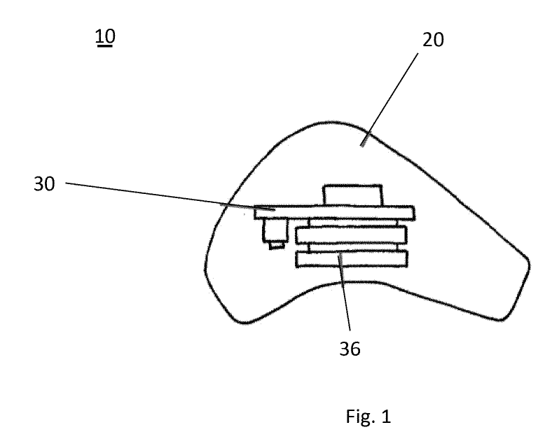

As illustrated in FIGS. 1 through 4D, the anxiety control device 10 includes an outer housing 20, a vibration unit 30, and electronic components. The outer housing 20 may be sized and shaped to fit ergonomically and substantially within a hand of the human user. The vibration unit 30 may be any other device configured to convert current into vibration. The vibration unit 30 may include, but is not limited to a tactile transducer, a linear transducer, a haptic element. However, the vibration unit 30 is broader may include devices or mechanisms other than and in addition to vibration units as described in the present disclosure. In alternative embodiments, however, a variety of emission techniques may be used with the vibration unit 30. For example, the devices as described herein may be configured to use electrical, photic, and/or audio stimulation to achieve the desired brainwave synchronization. In some instances, the vibration unit may be referred to as a stimulation unit or stimulation element.

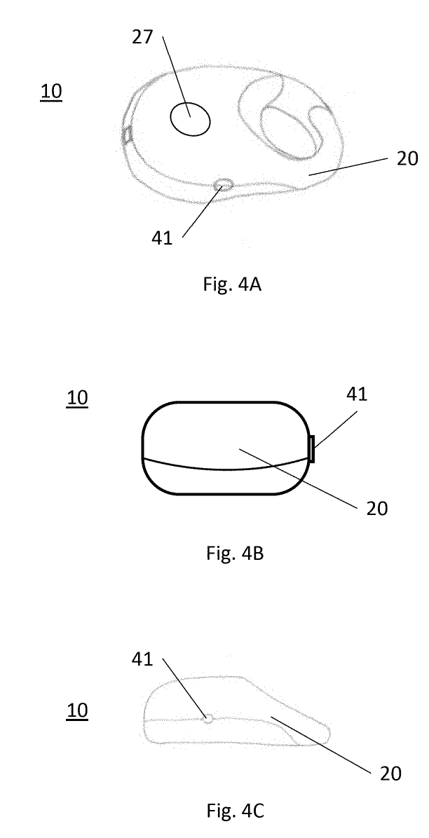

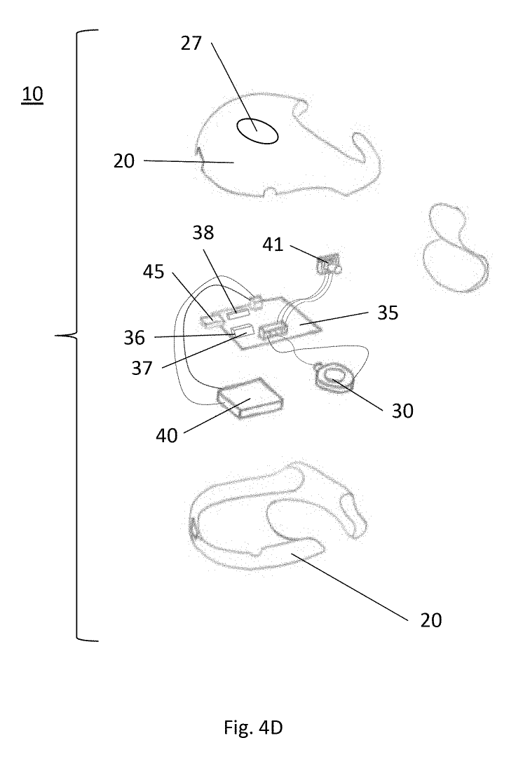

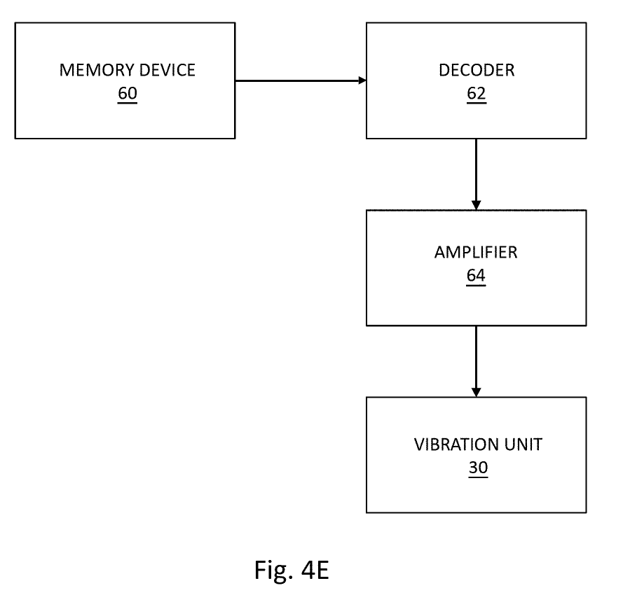

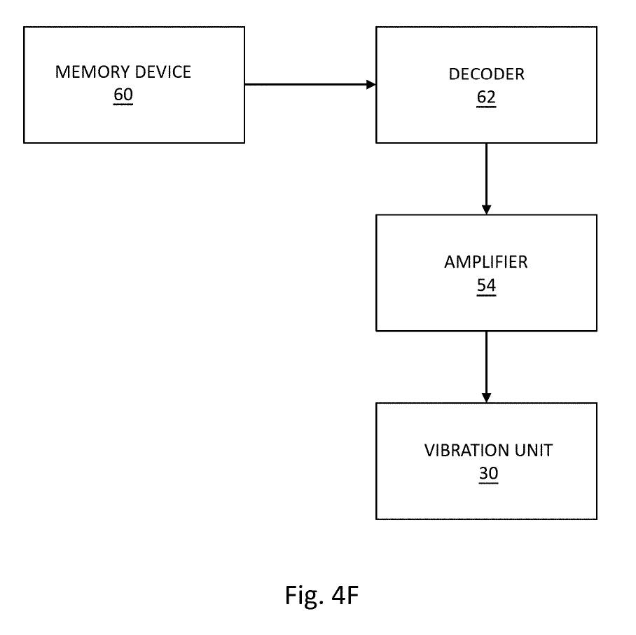

The electronic components include a controller 36. The controller 36 may include a memory 37, a processor (not shown), a communications unit 38, and an optional amplifier 54 (not shown). The controller 36, which may be a microcontroller, is electronically coupled to the communications unit 38. The communications unit 38 may be a transmitter, receiver, a transmitter/receiver, or a transceiver, or communications bus typical in electronic circuitry that perm communications and/or signal transmission between various electronic components. The device 10 may include any suitable power source 40. For instance, as illustrated in FIG. 4D, the device may be powered by a rechargeable battery rechargeable battery 10, the device may have a recharging port 45. Alternatively, the device 10 may include one or more removable batteries. The electronic components may be carried on a printed circuit board (“PCB”) 35.

In operation, the anxiety control device 10 reads the waveform signals 70 stored as data files 71 in the memory 37 of the controller 36. For example, the data files may be audio files such as a waveform audio file format (WAV), e.g. .wav files, or an audio interchange file (AIFF) or any other audio file format. One of ordinary skill in the art having read this specification will understand that all types of data files may be used. In one example, any data file may be used that is able to be used to generate a waveform. In a further embodiment, a data file may be used if it generates desired relative differences between successive pulses in a waveform. The controller 36 reads the data file and sends a signal to the vibration unit 30to emit the desired type of stimulation. The device 10 then transmits or imparts a form of the waveform signal 70 to the user through the vibration unit 30.

In certain embodiments, the housing 20 may include an opening 27 within the housing 20 that allows for direct contact between the vibration unit 30 and the user's skin. Such direct contact provides the most effective means of imparting the desired frequency signal to the user. In alternative embodiments (not shown), the housing 20 may not include an opening 27, but instead may have a thinner section proximate to the stimulation 30, or may have a thin, flexible material that covers the opening 27 but still allows for the frequency signal to be effectively transmitted to the user's cranium.

In use, the device 10 is positioned on the user's head such that the user's brain state or brain waves tend to follow signals generated by the device when powered on, thereby achieving altered brain states using a variety of device embodiments and signals.

Signals imparted to the brain such that the brain tends to follow those signals or change brain states can be achieved through a variety of device embodiments and signals. In an embodiment of the device 10, the user places the device transmitting element 30 behind his or her ear, at the base of the head. A user can then activate the device 10 when they are experiencing increasing levels of stress, during an anxiety attack, or whenever they feel the need for an immediate clear mind. The user places the device 10 at or on the instructed location, turns the device on, and his or her brain state will quickly react to synchronize the user's brainwaves to the signals generated by the device 10. In other embodiments, the performance of the device 10 can be enhanced through various additional stimulation or emission methods, and/or various frequency imparting locations on the user.

The devices as described herein may use a variety of signals and waveforms to stimulate different brain states. The signal can be emitted at a range of amplitudes, frequencies, and pitch combinations to achieve different effects. The signal includes a pattern sequence comprised of a plurality of pulses, with each pulse defined as the interval between two adjacent troughs in a signal. In the present disclosure, the pattern sequence may be groups of pulses whereby each pulse within the group varies in terms amplitude, frequency or pitch over time. Furthermore, successive groups of pulses may vary in terms of amplitude, frequency or pitch such that one set of pulses are dissimilar from another set of pulses. Thus, while frequencies can vary, signal amplitudes and/or pitches within certain signal responses may vary. Alternatively, the amplitudes and/or pitches even among a series of signal responses may vary. The pattern sequence as described herein may be a repeating pattern sequence.

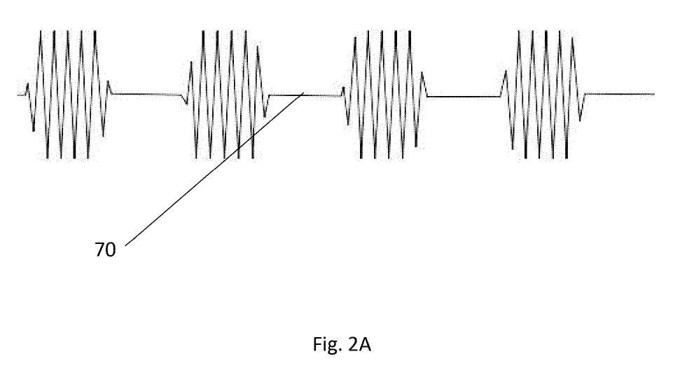

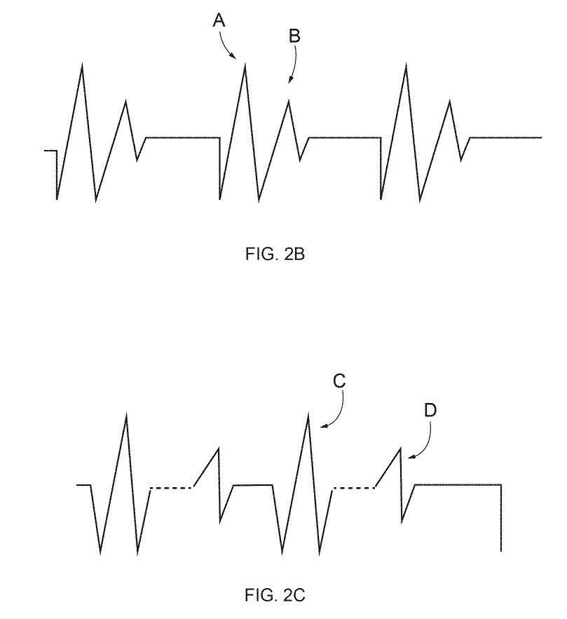

A few exemplary signals are illustrated in FIGS. 2A-2C. For instance, FIG. 2A illustrates a signal with a pattern sequence according to an embodiment of the present disclosure. As illustrated, the signal includes a series of pulse signals with each pulse having a predetermined frequency and amplitude. Both the frequency and amplitude within each signal pulse are similar among all the series of pulses in the signal. In other words, the signal may have consistent pulses throughout its signal. In another example of a signal, the signal may have two dominant frequencies and one primary amplitude. However, it should be appreciated that alternative signal patterns may be stored within device 10 that have a plurality of or varying frequencies and a plurality of amplitudes.

FIG. 2B illustrates a further exemplary signal that may be reproduced as a result of data filed stored in devices described herein. The signal illustrated in FIG. 2B comprises a repeating transition pattern sequence. The illustrated signal includes a first signal portion A and a second signal portion B, with each signal portion having a different amplitude than the other. As shown, the second signal portion B has a lower amplitude than signal portion A. While the signal shown in FIG. 2B illustrates two repeating portions, the signal may have more than two than two repeating portions. For example, the signal may have three repeating portions, four repeating portions, or even five repeating portions. Furthermore, in various instances, the pattern sequence may have one or more of various shapes including (but not limited to) square wave, sinewave, sawtooth, triangle, etc.

FIG. 2C illustrates a signal with two different repeating portions. As shown, the signal has a first amplitude portion C and a second amplitude portion D. The first amplitude portion C may be larger than the second amplitude portion D. However, what is illustrates is only exemplary as amplitude portion D may have higher amplitude than amplitude portion C. Also, as shown, there may be other amplitude variations between portion C and portion D. Furthermore, in another example, a repeating portion is amplitude variation that is repetitive as far as actual amounts of amplitude. In yet another example, the repeating portion is amplitude variation that is repetitive as far as relative amounts of amplitude, such that one amplitude is higher than another or one amplitude is lower than another. By using the word “relative” what is intended is that the amplitude of one pulse is higher or lower than the amplitude of another pulse. Thus, the actual amplitude of each pulse may be irrelevant; what is relevant is that in a repeating sequence of pulses, the amplitude of a pulse is higher or lower than in another pulse in a sequence. For example, in FIG. 2B, the amplitude of pulse B is less than the amplitude of pulse A, and the actual amplitude may or may not be important. In FIG. 2C, the amplitude of pulse D is less than the amplitude of pulse C. In a further exemplary embodiment of the present disclosure, the relative amplitude levels are independent of actual amplitude levels of the first pulse and the following pulses.

In an embodiment of the present disclosure, the pattern sequence is a plurality of pulses with each pulse having the same amplitude. In another embodiment of the present disclosure, the pattern sequence is a plurality of pulses having a first pulse at a first amplitude and a second pulse at a second amplitude that is different from than the first amplitude. In another embodiment of the present disclosure, the pattern sequence is a plurality of pulses having a first pulse at a first amplitude and a second set of pulses with amplitudes with the amplitudes in the second set of pulses being different from the first amplitude. In a such an example, the amplitude in first pulses are lower than the amplitude in the other pulses. Conversely, the amplitude levels in the first pulses are higher than the amplitudes in the other pulses.

In yet another embodiment of the present disclosure, the pattern sequence is a plurality of pulses with each pulse having the same frequency. In another embodiment of the present disclosure, the pattern sequence is a plurality of pulses having a first pulse at a first frequency and a second pulse at a second frequency that is different from the first frequency. In another embodiment of the present disclosure, the pattern sequence is a plurality of pulses having a first pulse at a first frequency and a second set of pulses with frequencies with the frequencies in the second set of pulses being different than the first frequency. In yet another embodiment of the present disclosure, the pattern sequence is a first set pulses and a second set of pulses, wherein the first set of pulses and the second set of pulses have a frequency that varies over a period of time. In such an embodiment, the frequency and/or amplitude with each set of pulses may vary or be similar. However, the frequency within successive sets of pulses may vary over time. In one example, the frequencies can range between 4 Hz to 40 Hz or even outside this range. In another example, the signal tone may have a frequency range between 4 to 8 Hz, consistent with theta waves. In another example, the signals can have frequency in the range of 12-40 Hz, consistent with beta waves. However, the frequencies can clearly fall outside of this range as needed.

In another embodiment of the present disclosure, the pattern sequence is a plurality of pulses with each pulse having the same pitch. In another embodiment of the present disclosure, the pattern of sequences is a plurality of pulses having a first pulse at a first pitch and a second pulse at a second pitch that is different from than the first pitch. In another embodiment of the present disclosure, the pattern sequence is a plurality of pulses having a first pulse at a first pitch and a second set of pulses with pitches with the pitches of the second set of pulses are different from the first pitch.

In another embodiment of the present disclosure, the pattern sequence is a plurality of pulses with each pulse having the same speed. In another embodiment, the pattern sequence is a plurality of pulses having a first pulse at a first speed and a second pulse at a second speed that is different from than the first speed. In another embodiment, the pattern sequence is a plurality of pulses having a first pulse at a first speed and a second set of pulses with speeds, wherein the speeds of the second set of pulses are different than the first speed. The speeds as used herein are broad and may range between 0.25 pulses per second up to 10 pulses per second, or even higher.

FIGS. 4E and 4F illustrate variations in how a data file is accessed and concerted to stimulation signals in the devices as described in the present disclosure. The embodiment shown in FIG. 4E allows the data the controls the vibration unit 30 to be stored in a computing device 50 that is physically separate from the housing 10 that contains the vibration unit 30. The embodiment of FIG. 4F allows the data that controls the vibration unit 30 to be in the same package as the vibration unit 30. One of ordinary skill will appreciate that either embodiment may be modified to include wired and wireless options.

Referring to FIG. 4E is a system is shown that includes devices described herein for providing tactile stimulation to the user. The system may include a computing device 50, a communications unit 38, an amplifier 54, and the vibration unit 30. As an example, the computing device 50, which may or not form part of the device 10, may include a memory unit, a processor, a communications unit, and an interface, as is typical and known in the computing arts. The computing device 50 may be a variety of different computing devices, for example PC, tablet, smartphone, etc. The memory unit includes stored data and/or executable instructions for controlling the vibration unit 30. Computing device 50 transmits data that controls vibration unit 30 to the communications unit 38, for example Bluetooth integrated circuit (IC) or coms bus. For instance, data may be transmitted via Bluetooth communications protocol. Although the communications unit 38 may be Bluetooth capable, other communications systems may be used. Bluetooth is merely exemplary as any form of wireless or wired communications may be used transmit data to the device 10. In any event, the communications unit 38 receives data from computing device 50 and converts the data to an analog signal. This analog signal may then be sent to an amplifier 54. An exemplary amplifier 54 is PAM8406 which is available from Diodes Incorporated. Amplifier 54 transmits the amplified analog signal to the vibration unit 30. The vibration unit 30 then provides stimulation to the part of the user's head that it is in contact with.

Referring to FIG. 4F, a system is shown that includes a memory device 60, decoder 62, a controller (not shown), optional amplifier 54, and the vibration unit 30. Data and/or executable code used to control the vibration unit 30 may be stored in the memory device 60. Memory device 60 may be a memory card and may be configured to permit data stored therein to be accessed by a decoder 62. An exemplary decoder 62 is an MP3 integrated circuit (IC), which may be a TF card MP3 decoder board GPD 2856A, which is available from Generalplus. The decoder 62 may decode the data and convey that data to the optional onboard amplifier. Alternatively, the amplifier 54 may form part of the device 10. As illustrated, the decoder 62 produces an analog signal, which is amplified by amplifier 54. The amplified output of the amplifier 54 is then transmitted to the vibration unit 30, wherein the vibration unit 30 operates in accordance with the data file.

Certain exemplary embodiments of the devices as described herein are configured to record various physiological parameters, device metrics, and usage data. Using communication networks as described elsewhere such data may be transmitted to or between the devices described herein and other computing devices, such as a smartphone, tablet or device, a database and the brain state influencing device 10. For example, the device 10 may be outfitted to incorporate biofeedback and electroencephalogram (“EEG”) data to allow for the reading or recording of the user's brainwaves. Through such reading of brainwaves, the device may be able to be adjusted, including by altering the imparted tone or transmission frequencies to achieve desired results. Based on the EEG readings, the devices can either send a notification to the user's phone when certain brainwave patterns are sensed, or automatically activate the device 10 to subdue unwanted brain states preemptively. The device in certain embodiments can use a variety of equipment to measure brainwaves, as well as other physiological metrics, including sweat output, muscle tension, respiration rate, and heart rate. The devices in certain embodiments can use a variety of sensors to measure various physiological metrics, including brainwaves, perspiration, respiration rate, and/or heart rate. In a further exemplary embodiment of the present disclosure, the vibration unit may be actuated in response to detection of one or more predetermined brainwave patterns from the brain of the patient that is receiving the tactile stimulation.

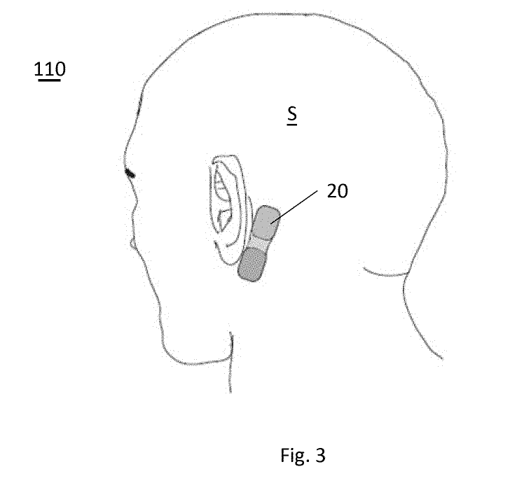

As shown in FIG. 3, another embodiment of a device 110 can be placed behind the ear at the base of the head to allow the vibrations to transmit directly through bone conduction. The device 110 shown in FIG. 3 has similar components to the device 10 shown in FIGS. 1 and 4A-4D and the same reference numbers are used to identify features that are common between the device 10 and device 110. It should be appreciated that device 110 can also be placed on other parts of the head as long as the pattern sequences are being properly imparted to and decoded by the user's brain.

In use, the individual is able to place and maintain the device 110 in contact with or adjacent to his or her head with various orientations. In one embodiment device 110 is held in place with force from a person's hand. In another embodiment an artificial structure (such as a hat or netting) holds device 110 in place. the device 110 placed behind the ear (or on the head), the vibrations bypass the eardrum and signals are imparted directly to the cochlea. This will allow users to not only feel the vibrations, but also audibly hear the signals. In such a configuration, the individual may optionally block the ear canal of his or her respective ear that the device is behind, which will create an intense and immersive audible effect. The device can emit a number of different signals with pattern sequences, audibly and palpably, thereby increasing the effectiveness of inducing brainwave synchronization. In a further exemplary embodiment of the present disclosure, the vibration unit 30 may be a tactile transducer that provides tactile stimulation to a person's head while the device 110 is held against the head.

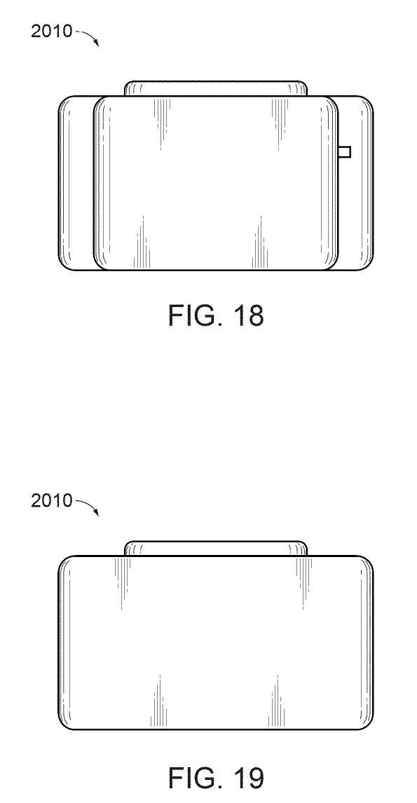

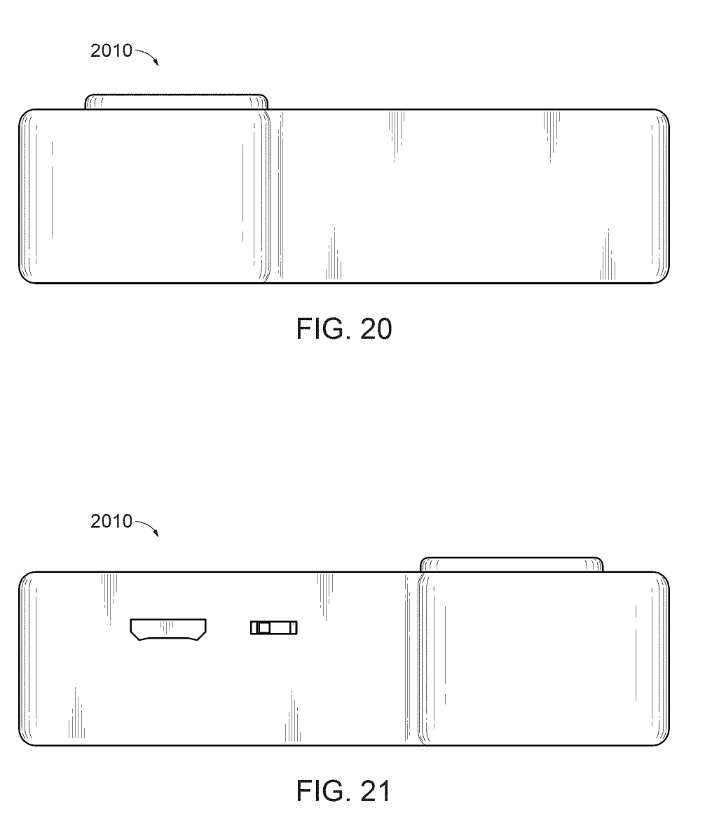

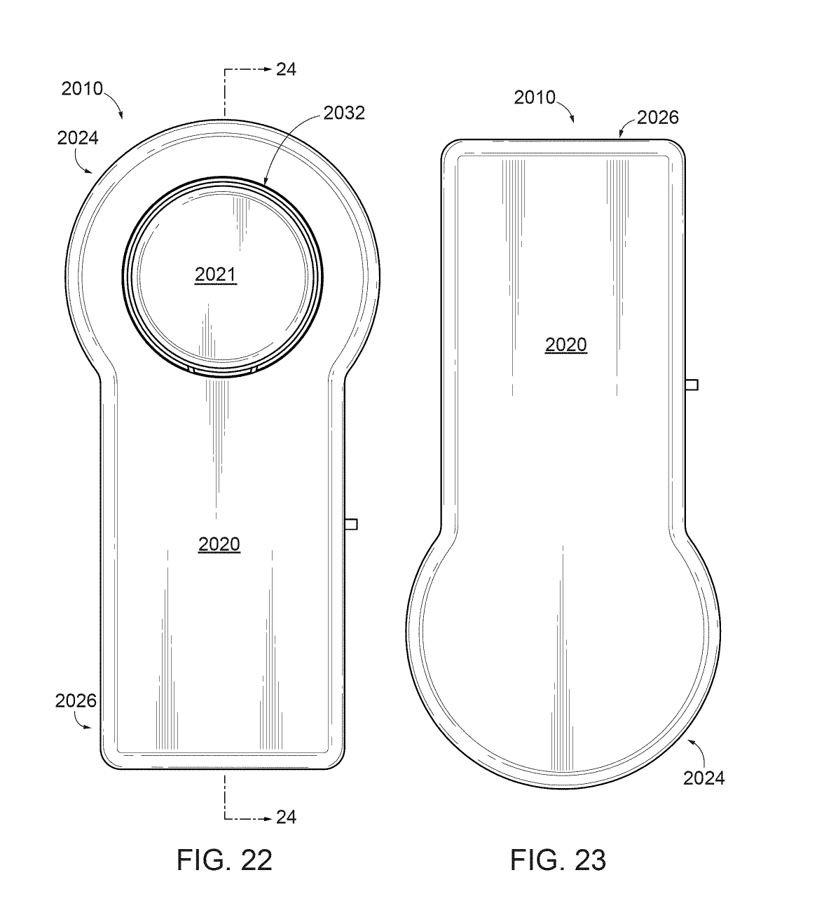

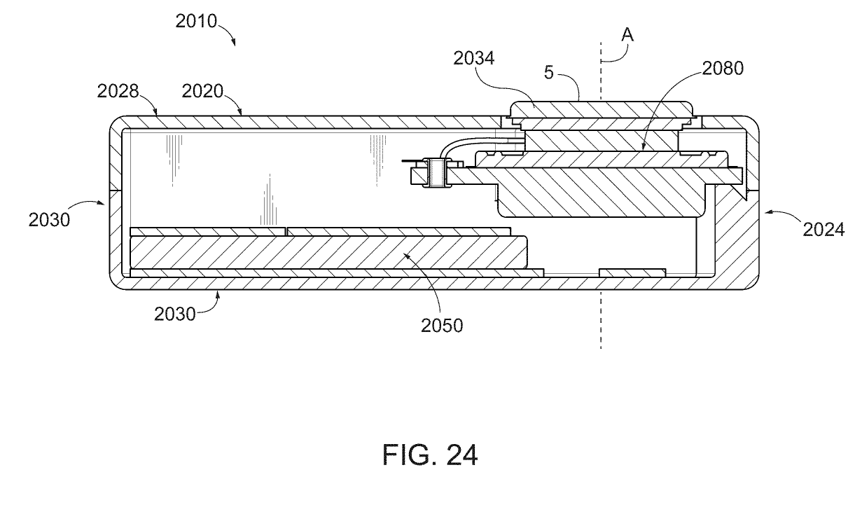

As also shown in FIGS. 3 and 10 through 24, the devices as described herein can be compact and portable. The overall size of the device has been minimized to improve its compact nature and fit ergonomically in the hand of the user. Such a compact configuration allows users to operate and use the device discreetly. Often times, anxiety attacks and intense episodes of stress do not occur in the comfort of an individual's home, but rather at work, public or social events and other various settings where the individual is not alone and comfortable. An individual will be able to readily carry the device in their pocket or any type of bag. This allows users to address the need to counter an anxiety attack and de-escalate stress wherever they may be, and not having to worry about being able to address an involuntary anxiety and high stress attack.

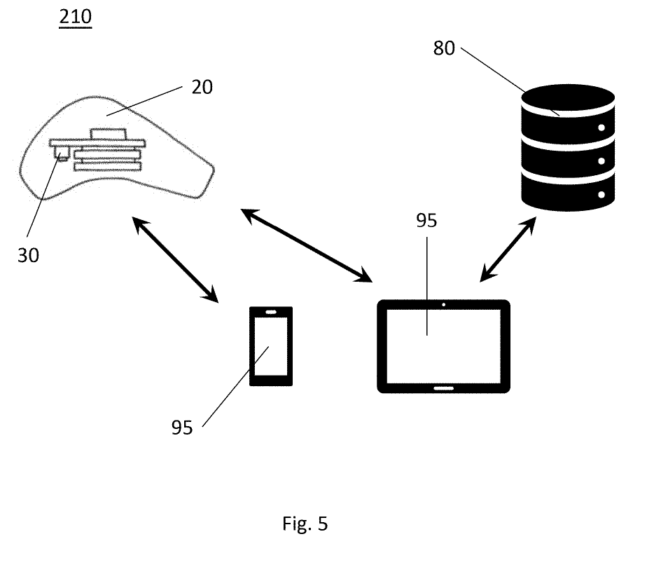

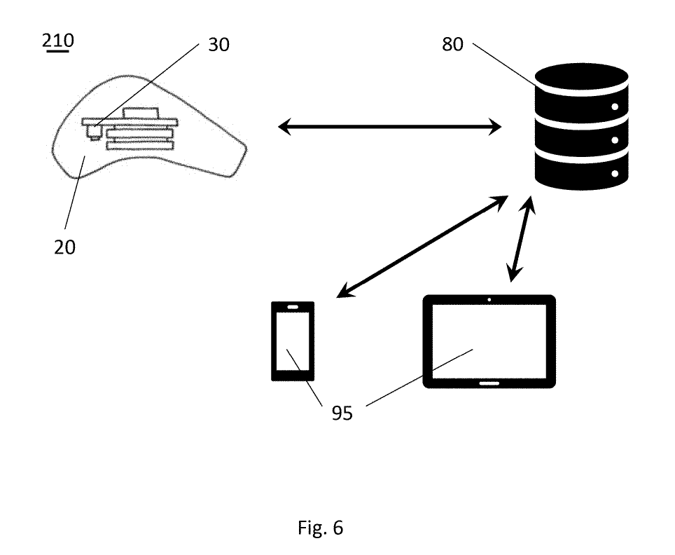

FIGS. 5 and 6 illustrate embodiments of the device 210 configured to interface with one or more computing devices over a network. The device 210 shown in FIGS. 5 and 6 have similar components to the device 10 shown in FIGS. 1 and 4A-4D and the same reference numbers are used to identify features that are common between the device 10 and device 210. For instance, the device 210 includes a controller, a communications unit 38, and a vibration unit 30. The device 210 illustrated in FIGS. 5 and 6 is configured to transmit, or transmit and receive the recorded data through a variety of means. For example, the device 210 may record the data and send it to a remote computing device 95, such as the user's phone, tablet or other device 95, via a communications network, e.g. using a Bluetooth protocol. The data may then be stored in a database 80 where it is stored for later access or analysis. The computing device 95 may also receive relevant data from the device 210, and/or send relevant data to the device 210 through known two-way signal communication protocols, such as a Wi-Fi or Bluetooth protocols. The database 80 may also be communicatively connected with the user's phone to allow the user to view the trends and data in a user-friendly fashion. In further embodiments, the device 210 may be configured in other architectures with additional communication components that alternatively allow the data to be recorded and stored, and later displayed to the user. The device 210 may also include additional sensors configured to generate data about the user, e.g., EEG and/or other physiological data.

In a further embodiment, the device 10 may also include a communications unit 38 capable of transmitting data gathered by the device 10 to a remote computing device, which may include a database. The communications unit 38 will record when the device is turned on, the duration of use, as well as other metrics or factors to record trends and patterns. The user will be able to download an external application on a smartphone, tablet or visit a website to view the recorded data and metrics. The data generated from these sensors can be synched with remote computing device, such as the user's phone, tablet or other device, to monitor the user's metrics. All of this information will be displayed on the remoting computing device via graphical user interface.

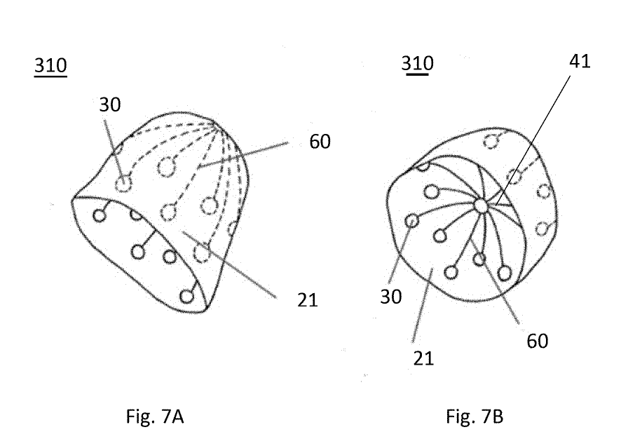

The devices as described herein may be implemented as wearable apparel. In one such a configuration, as shown in FIGS. 7A and 7B, the device 310 is implemented as a headwear article 21. The device 310 includes a plurality of frequency emitting elements 30 that are spread out throughout the apparel in a “spider web” fashion. As illustrated, the device 210 includes a plurality of flexible arms 60 and frequency emitting elements 30 disposed on one or more of the flexible arms 60. The arms 60 are integrated into the headwear article 21 are flexible enough to bend and conform to the user's head when the article 21 is placed over their head. The flexible nature of the arms 60 allows for the movement and positioning of any of the frequency emitting elements 30. The device 10 can also be incorporated into any material or specific apparel. With this configuration, the user can wear the device to ensure ease of use and accessibility. In the device 310 illustrated in FIGS. 7A and 7B, the user is able to select which nodes, areas, or both that emits stimulation. The user is also able to choose if they want stimulation at one single frequency, or to have the stimulation at varying frequencies, intensities and patterns. For example, a user could choose to have a “wave” or “sweeping” pattern of stimulation, standalone or in addition to other patterns. Other patterns may include music. Thus, while in one example, a tactile transducer is used to provide tactile stimulation, traditional aural stimulation (i.e. binaural beats, music, guided meditation/voice or a combination of these) may be provided as well. A user can also choose to have the stimulation constant, with or without specific pattern sequences. In an exemplary embodiment of the present disclosure, the tactile transducer provides a pulse of energy to a large surface area, directed at the mastoid part of the temporal bone. Rather than targeting one precise location, the exemplary device disperses energy to the mastoid part of the temporal bone through a “shotgun approach.” In an exemplary embodiment of the present disclosure, the tactile transducer thus also provides aural stimulation.

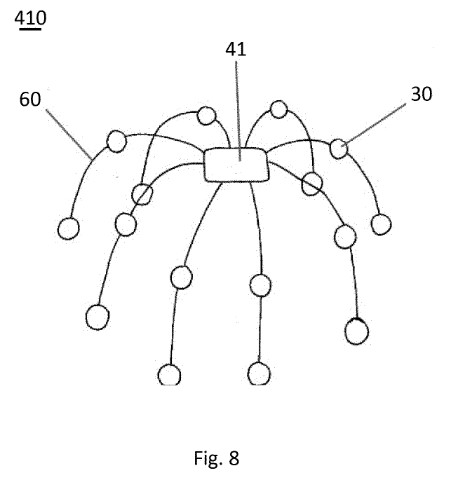

Similar to the device configuration illustrated in FIGS. 7A and 7B, the “spider web” configuration of the system may also be worn without being incorporated into a hat or apparel. The configuration seen in FIG. 8 shows the device 410 in the webbed system which can be placed on a user's head. The device 410 shown in FIG. 8 has similar components to the device 310 shown in FIG. 7 except that the device is not implemented in headwear. However, the same reference numbers are used to identify features that are common between the device 410 and device 310. In the embodiment shown, the user can manipulate the flexible arms 60 to choose where each stimulation element 30 is placed to have the greatest effect. Stimulation elements 30 are capable of delivering a variety of emission signals. The device 410 also includes a control panel 41 at the top of the device 410 where the user can turn the device on and off, as well as manipulate one or more of the settings that have been previously described. The device 410 may also be placed or integrated into any hat, headwear or any other apparel.

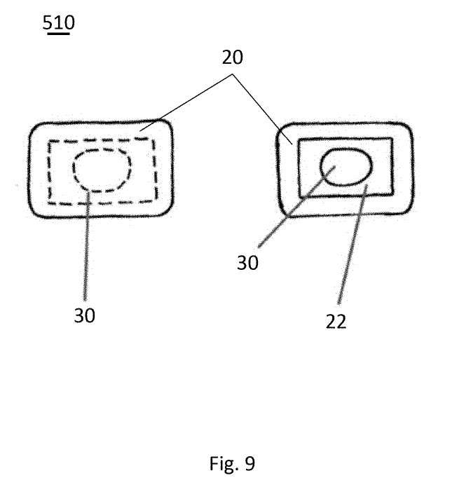

Referring to FIG. 9, another embodiment of the present disclosure includes a device 510 with an adhesive material 22 that is attachable to the user. The device 510 shown in FIG. 9 has similar components to the device 10 shown in FIGS. 1 and 4A-4D and the same reference numbers are used to identify features that are common between the device 10 and device 510. The 510 will include a PCB 35, battery 40 and internal components necessary to operate the device 510 and at least one frequency emitting node 30. In this embodiment, adhesive material 22 is used to stick the respective nodes to any surface. With the device 510, the user can choose to place a node 30 either directly on their body, or in any apparel that the user may choose to wear. With this configuration, the user can be specific about where they want to place the frequency emitting element 30, as well as how many elements 30 that they would like to use. As with other devices described in the present disclosure, the device 510 may be programed to achieve different stimulation sequences throughout the nodal system such as sweeping pulses or constant stimulation in addition to various stimulation methods capable within the other configurations.

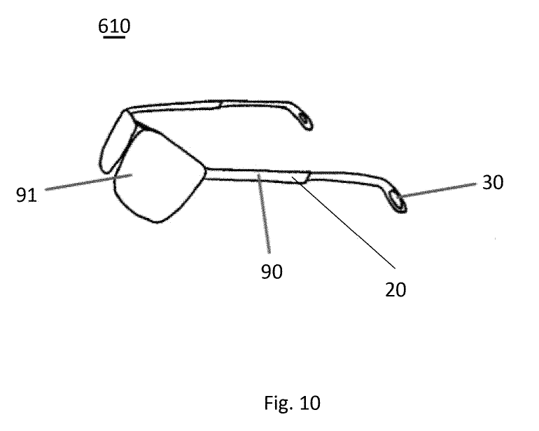

In still a further embodiment, as shown in FIG. 10, a device 610 can be incorporated as eyewear 90, such as eyeglasses or sunglasses. The device 610 shown in FIG. 10 has similar components to the device 10 shown in FIGS. 1 and 4A-4D and the same reference numbers are used to identify features that are common between the device 10 and device 610. The device 610 includes an eyeglass frame with a lens rim 91, a left temple arm, and a right temple arm that are configured to extend to the left and right ears, respectively. In the device 610, the stimulation elements 30 are located at the distal end of each temple arm of the eyewear 90. There may also be stimulation elements 30 integrated around the lens rim 91 that can also transmit photic and other stimulation. The electronic components that operate the system may be integrated into the frame.

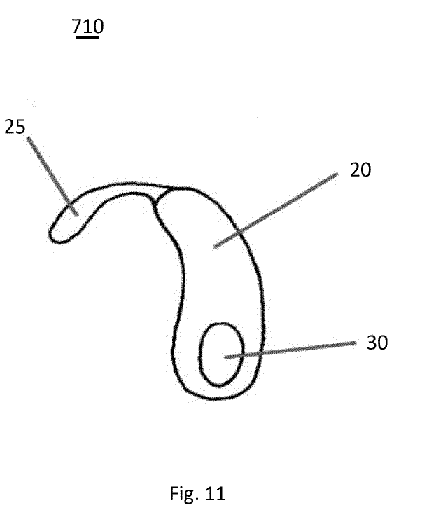

As shown in FIG. 11, another embodiment of a device 710 is configured to be worn over the ear, similar to a hearing aid. The device 710 shown in FIG. 11 has similar components to the device 10 shown in FIGS. 1 and 4A-4D and the same reference numbers are used to identify features that are common between the device 10 and device 710. The device 710 includes a housing 20 and at least one frequency emitting element 30. The housing 20 includes a base and a flexible arm 25 that allows the user to fasten the device 710 around his or her ear comfortably while being securely positioned in place. The simulation node 30 is integrated into the base of the device that fits behind the user's ear. The electronic components that operate the system are stored within the enclosed portion of the housing 20.

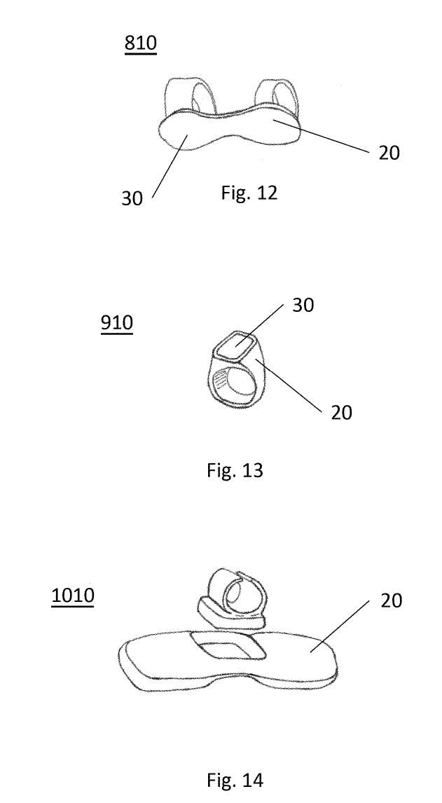

The embodiments shown in FIGS. 12 through 16 illustrate that the devices described herein may be fabricated various alternative sizes and configurations. For example, as shown in FIGS. 12 and 13, the device may be very discreetly configured to be a ring 810 or finger type device that the user can easily place on his or her finger, and then simply position his or her hand and the device 810 behind their ear. The device 810 shown in FIGS. 12 and 13 have similar components to the device 10 shown in FIGS. 1 and 4A-4D and the same reference numbers are used to identify features that are common between the device 10 and device 810. In a further variation shown in FIG. 14, a device 910 removably attached to a ring element, which is formed to fit the housing as shown in FIG. 14. Again, the device 910 shown in FIG. 14 has similar components to the device 10 shown in FIGS. 1 and 4A-4D and the same reference numbers are used to identify features that are common between the device 10 and device 910.

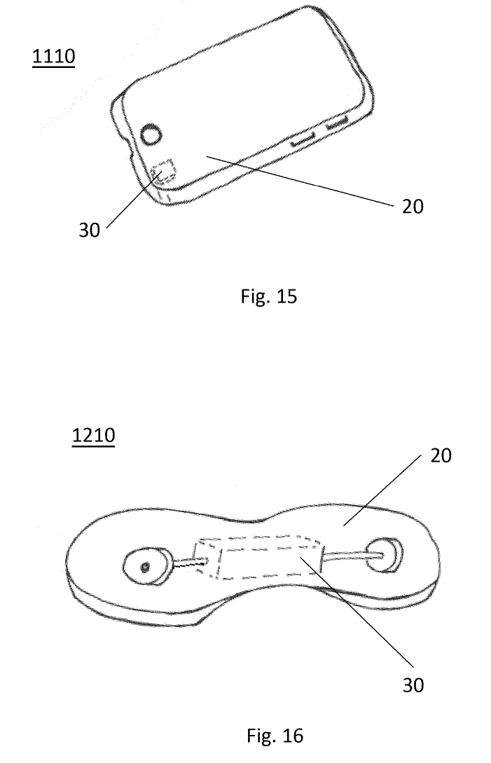

As shown in FIG. 15, the device 500 may be configured to be incorporated into a smartphone case, such that the smartphone case forms the housing 20 and also include other components, such as the vibration unit 30, controller 36, and power source 40. The device 500 shown in FIG. 15 has similar components to the device 10 shown in FIGS. 1 and 4A-4D and the same reference numbers are used to identify features that are common between the device 10 and device 500. In use, the user can position and rest the smartphone case behind the user's ear and switch the device on. As an alternative to having a separate case with a vibration unit 30, a software application when executed, may control operation of the vibration unit 30 in order to generate the desired frequency signals. In such an embodiment, the user can similarly position his or her smartphone behind his or her ear to impart the desired frequency signals and thereby control any anxiety attacks.

In yet another embodiment, a computing device may include, stored in its memory, a software application that is executable by a processor of the computing device, when executed, may control operation of the vibration unit 30 in the device 10 (or others) that is linked with the computing device, in order to generate the desired frequency signals. In the software application described above, when utilizing the software application and with the device being in an on state, the application pushes a notification to the user's phone (or whichever device is synched) and asks the user if they are experiencing or had an anxiety attack. The user is then able to record, if known, why the attack occurred, and record notes that may be relevant or important to the attack. The data is stored and displayed in a user-friendly format to show how frequently an individual is having attacks/stress as well as in what environments such conditions are manifested. With such data and analytics, the user may be able to recognize certain trends and then use this information to help prevent further anxiety attacks/stress. Furthermore, clinical professionals such as psychologists and psychiatrists, as well as parents, caretakers and other authorized recipients may also be able to download the application to view the user's data. Clinical professionals or caretakers can then review the data to determine any trends or other valuable data to gain a better insight on the user's condition, and then may be able to structure more personalized treatment, coping methods, and focused therapy sessions. All of this data will be recorded and sent through the communications elements in the devices described herein.

As described, in various embodiments, the device can produce various ranges of frequencies, amplitudes, and pitches. Moreover, as illustrated and disclosed above, the device may be fabricated or configured into a wide variety of shapes, sizes, and configurations. A variety of interchangeable stimulation nodes can be utilized to impart the desired stimuli to the user. The device is compact and discreet to allow the individual to achieve desired brainwave synchronization.

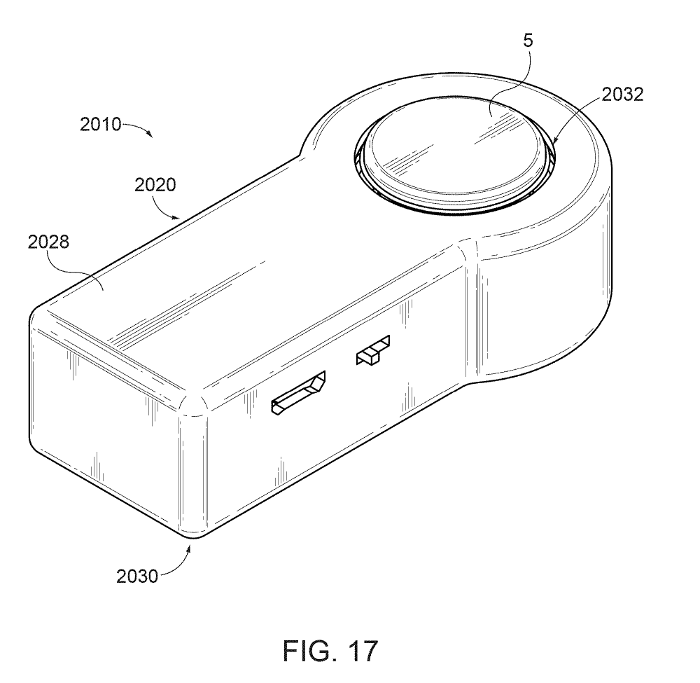

FIGS. 17-24 illustrate yet another embodiment the present disclosure is a handheld device 2010. The handheld device 2010 is configured to provide tactile stimulation to a head of a human user and includes an outer housing 2020, a controller 2050, and a vibration unit 2080.

Continuing with FIGS. 17-24, the outer housing 2020 may be sized and shaped to fit ergonomically and substantially within a hand of the human user. As with other embodiments, the outer housing 2010 defines an internal space 2022 and skin facing surface S for placement against the user's skin. In the example illustrated in FIGS. 17-24, the outer housing 2020 has a first end 2024, a second end 2026 opposite the first end 2024, a top housing component 2028, a bottom housing component 2030 opposite the top housing component 2028, and an opening 2032 defined by the bottom housing component 2030. The bottom housing component 2028 is configured to face the head of the user. As shown, the vibration unit 2080 is aligned with the opening so that vibrations emanate from the housing 2020 proximate the opening 2032. More specifically, the vibration unit 2080 is aligned with the skin facing surface 2021 of the outer housing 2020 along an axis A that is substantially perpendicular to the outer housing 2020. In some instance, the handheld device further comprises a cover 2034 disposed in the opening 2032 and adjacent to the vibration unit 2080. However, neither the opening nor the cover are essential; the housing can completely enclose the components of the device 2010.

Still referring to FIGS. 17-24, the controller 2050 may be contained within internal space of the outer housing. The controller 2050 includes a processor, a memory unit, and a communications unit. The memory includes stored thereon a data file. As described above, the data file includes a pattern sequence. The processor is configured to generate a signal corresponding to the pattern sequence contained within the data file. In addition, the device 2020 may include an optional amplifier (not numbered) in series with the vibration unit. The amplifier is configured to amplify the signal from the processor. In certain embodiments, the device may include just a controller, such as a processor. In such an example, the data file and/or executable codes can be on a remote computing device and transmitted to the controller for execution to thereby activate the vibration unit.

Continuing with FIGS. 17-24, the vibration unit 2080 is in electronic communication with the processor and communications unit of the controller 2050. As with other embodiments described in the present disclosure, the vibration unit 2080 is configured to be actuated in response to a signal from the processor. More specifically, the vibration unit 2080 generates vibrations that correspond to the pattern sequence when the controller is activated. When the skin facing surface of the handheld device is placed in contact with the head of user and the controller is activated, the vibration unit stimulates the user head as described elsewhere in the present disclosure. In one example, the vibration unit 2080 is actuated responsive to detection of one or more predetermined brainwave patterns in the user. As illustrated, the vibration unit 2080 is a tactile transducer configured to convert a current into mechanical vibrations that correspond to the pattern sequence. In some examples, the tactical transducer includes a responsive surface that generates the mechanical vibrations when actuated by the processor. It should be appreciated that the vibration unit can generate any of the signals and patterns described in the present disclosure.

The embodiment illustrated in FIGS. 17-24 may employ any of the signal response described herein. In particular, the device 2010 may generate a pattern sequence. The pattern sequence is a plurality of pulses with each pulse having the same amplitude. In another embodiment of the present disclosure, the pattern sequence is a plurality of pulses having a first pulse at a first amplitude and a second pulse at a second amplitude that is different from than the first amplitude. In another embodiment of the present disclosure, the pattern sequence is a plurality of pulses having a first pulse at a first amplitude and a second set of pulses with amplitudes with the amplitudes in the second set of pulses being different from the first amplitude. In a such an example, the amplitude in first pulses are lower than the amplitude in the other pulses. Conversely, the amplitude levels in the first pulses are higher than the amplitudes in the other pulses.

In yet another embodiment of the present disclosure, the pattern sequence generated by the device 2010 is a plurality of pulses with each pulse having the same frequency. In another embodiment of the present disclosure, the pattern sequence is a plurality of pulses having a first pulse at a first frequency and a second pulse at a second frequency that is different from the first frequency. In another embodiment of the present disclosure, the pattern sequence is a plurality of pulses having a first pulse at a first frequency and a second set of pulses with frequencies with the frequencies in the second set of pulses being different than the first frequency. In yet another embodiment of the present disclosure, the pattern sequence is a first set pulses and a second set of pulses, wherein the first set of pulses and the second set of pulses have a frequency that varies over a period of time. In such an embodiment, the frequency and/or amplitude with each set of pulses may vary or be similar. However, the frequency within successive sets of pulses may vary over time. In one example, the frequencies can range between 4 Hz to 40 Hz or even outside this range. In another example, the signal tone may have a frequency range between 4 to 8 Hz, consistent with theta waves. In another example, the signals can have frequency in the range of 12-40 Hz, consistent with beta waves. However, the frequencies can clearly fall outside of this range as needed.

In another embodiment of the present disclosure, the device 2010 may generate a pattern sequence is a plurality of pulses with each pulse having the same pitch. In another embodiment of the present disclosure, the pattern of sequences is a plurality of pulses having a first pulse at a first pitch and a second pulse at a second pitch that is different from than the first pitch. In another embodiment of the present disclosure, the pattern sequence is a plurality of pulses having a first pulse at a first pitch and a second set of pulses with pitches with the pitches of the second set of pulses are different from the first pitch.

Another embodiment of the present disclosure is a method of providing stimulation to a head of a user. The method includes placing a skin facing surface of a handheld device proximate a head of a user. The method may include powering the handheld device to activate a controller contained within the handheld device. In one example, the method may include allowing access to a data file in memory of a controller, wherein the data file includes data that corresponds to a pattern sequence. The method may include, in response to accessing the data file, generating a signal corresponding to the pattern sequence. Furthermore, the method may include holding the handheld device in place proximate the head of the user so that a vibration unit in electronic communication with the controller generates vibrations that corresponds to the pattern sequence, thereby stimulating the user's head in accordance with the pattern sequence.

In addition to addressing anxiety and stress levels, the devices described herein have the capability to combat many different non-desired conditions, discomfort levels, or non-desired states of mind.

While several preferred embodiments and features of the inventive devices and systems for proactively influencing brainwave states have been described and disclosed, in particular with reference to the attached figures and drawings showing certain exemplary embodiments that relate to a particular embodiments and system components, such exemplary embodiments as shown are not to be construed as limiting the scope of the inventive device or systems. More particularly, as exemplified by the above described embodiments, alternative embodiments and configurations may be created that allow the user to discreetly use the device to impart a form of periodic frequency stimulation to influence the user's brainwave state to achieve a non-anxiety, non-elevated stress level condition. Moreover, alternative means of providing the frequency transmitting signal to the user may be incorporated into the device. While certain forms of vibration units have been disclosed and shown, alternative elements, such as a solenoid, as illustrated in FIG. 16, may be equally effective for operation and use.

The present disclosure relates to devices, systems and methods that allow users to discretely and directly reduce or alleviate anxiety attacks through proactive influencing of brainwave activity.

The devices described herein induces brainwave synchronization and eliminates or reduces the effects of an anxiety attack. In certain desired embodiments, the device uses pattern sequences to induce a frequency following response in the brain, which in turn synchronizes the user's brainwaves to the frequency of pattern sequences encoded in the vibration signals. In other aspects, the device also provides the user with an associated physical vibration to focus on and act as an anchor as part of the mechanism to control the anxiety attack.

The devices and methods a described herein allows individuals to substantially reduce or eliminate anxiety attacks, subdue the “fight or flight” response, and achieve other desired brain wave states. The device may also be effectively used during high stress situations that require precise, clear, and rapid critical thinking, such as, by way of example, law enforcement, commercial and military aviation personnel, professional athletes, and medical personnel. The device is also able to alleviate symptoms of many conditions by using frequency tone and signal stimulation through various methods including vibrational, electrical, photic, audial and other related methods. The device in different embodiments can emit the signal to the user in different ways. In various embodiments, the device may also incorporate bio-feedback and electroencephalogram (“EEG”) capabilities.

It will be recognized by those skilled in the art that other modifications, substitutions, and/or other applications are possible and such modifications, substitutions, and applications are within the true scope and spirit of the present disclosure. It is likewise understood that the attached claims are intended to cover all such modifications, substitutions, and/or applications.

What is claimed is:

1. A handheld device configured to provide tactile stimulation to a head of a human user, the handheld device comprising:
an outer housing sized and shaped to fit ergonomically and substantially within a hand of the human user, the outer housing defining an internal space and skin facing surface for placement adjacent to or proximate the user's head;
a controller contained within internal space of the outer housing, wherein the controller is configured to generate a signal corresponding to a pattern sequence; and
a vibration unit in electronic communication with the controller, the vibration unit being configured to be actuated in response to a signal from the controller so as to generate vibrations that correspond to the pattern sequence.

2. The handheld device according to claim 1, wherein the pattern sequence is one of:
a plurality of pulses with each pulse having the same amplitude;
a plurality of pulses having a first pulse at a first amplitude and a second pulse at a second amplitude that is different from than the first amplitude; or
a plurality of pulses having a first pulse at a first amplitude and a second set of pulses with amplitudes, wherein the amplitudes in the second set of pulses are different from the first amplitude in the first pulse.

3. The handheld device according to claim 1, wherein the pattern sequence is one of:
a plurality of pulses with each pulse having the same frequency;
a plurality of pulses having a first pulse at a first frequency and a second pulse at a second frequency that is different from than the first frequency;
a plurality of pulses that alternate in succession between a first pulse at a first frequency and a second set of pulses with frequencies, wherein the frequencies in the second set of pulses are different than the first frequency of the first pulse; or
a first set pulses and a second set of pulses, wherein the first set of pulses and the second set of pulses have a frequency that varies over a period of time.

4. The handheld device according to claim 1, wherein the pattern sequence is one of:
a plurality of pulses with each pulse having the same pitch;
a plurality of pulses having a first pulse at a first pitch and a second pulse at a second pitch that is different from than the first pitch; or
a plurality of pulses having a first pulse at a first pitch and a second set of pulses with pitches, wherein the pitches of the second set of pulses are different than the first pitch.

5. The handheld device according to claim 1, wherein the controller includes a processor, a memory unit, and a communications unit, the memory unit including stored thereon a data file, the data file including the pattern sequence, the processor being configured to generate the signal corresponding to the pattern sequence contained within the data file.

6. The handheld device according to claim 1, wherein the controller includes memory, the memory including stored thereon a data file, wherein the data file includes the pattern sequence.

7. The handheld device according to claim 1, wherein the vibration unit is a tactile transducer configured to convert a current into mechanical vibrations.

8. The handheld device according to claim 7, wherein the tactical transducer includes a responsive surface that generates the mechanical vibrations when actuated.

9. The handheld device according to claim 1, wherein the outer housing defines an opening at the skin facing surface, wherein the vibration unit is aligned with the opening.

10. The handheld device according to claim 9, further comprising a cover disposed in the opening and adjacent to the vibration unit.

11. The handheld device according to claim 1, wherein the vibration unit is aligned with the skin facing surface of the outer housing along an axis that is substantially perpendicular to the outer housing.

12. The handheld device according to claim 1, wherein the outer housing has a first end, a second end opposite the first end, a top housing component, a bottom housing component opposite the top housing component, and an opening defined by the bottom housing component, and the bottom housing component being configured to face the head of the user, wherein the vibration unit is aligned with the opening so that vibrations emanate from housing proximate the opening.

13. The handheld device according to claim 12, wherein the vibration unit is a tactile transducer configured to convert a current into mechanical vibrations.

14. The handheld device according to claim 12, wherein the vibration unit is a linear transducer.

15. The handheld device according to claim 12, wherein the vibration unit is a haptic element.

16. The handheld device according to claim 1, wherein the vibration unit is actuated responsive to detection of one or more predetermined brainwave patterns in the user.

17. A handheld device configured to provide tactile stimulation to a head of a human user, the handheld device comprising:
an outer housing sized and shaped to fit ergonomically and substantially within a hand of the human user, the housing defining an internal space and skin facing surface for placement adjacent to or proximate the user's head;
a controller contained within internal space of the outer housing, the controller including a processor and a memory unit, the memory including stored thereon a data file, the data file including a pattern sequence, the processor being configured to generate a signal corresponding to the pattern sequence contained within the data file; and
a vibration unit in electronic communication with the processor of the controller, the vibration unit is configured to be actuated in response to a signal from the processor so as to generate vibrations that correspond to the pattern sequence.

18. The handheld device according to claim 17, wherein the pattern sequence is one of:
a plurality of pulses with each pulse having the same amplitude;
a plurality of pulses having a first pulse at a first amplitude and a second pulse at a second amplitude that is different from than the first amplitude; or
a plurality of pulses having a first pulse at a first amplitude and a second set of pulses with amplitudes, wherein the amplitudes in the second set of pulses are different from the first amplitude in the first pulse.

19. The handheld device according to claim 17, wherein the pattern sequence is one of:
a plurality of pulses with each pulse having the same frequency;
a plurality of pulses having a first pulse at a first frequency and a second pulse at a second frequency that is different from than the first frequency;
a plurality of pulses that alternate in succession between a first pulse at a first frequency and a second set of pulses with frequencies, wherein the frequencies in the second set of pulses are different than the first frequency of the first pulse; or
a first set pulses and a second set of pulses, wherein the first set of pulses and the second set of pulses have a frequency that varies over a period of time.

20. The handheld device according to claim 17, wherein the pattern sequence is one of:
a plurality of pulses with each pulse having the same pitch;
a plurality of pulses having a first pulse at a first pitch and a second pulse at a second pitch that is different from than the first pitch; or
a plurality of pulses having a first pulse at a first pitch and a second set of pulses with pitches, wherein the pitches of the second set of pulses are different than the first pitch.

21. The handheld device according to claim 17, wherein the controller includes memory, the memory including stored thereon a data file, wherein the data file includes the pattern sequence.

22. The handheld device according to claim 17, wherein the vibration unit is a tactile transducer configured to convert a current into mechanical vibrations.

23. The handheld device according to claim 22, wherein the tactical transducer includes a responsive surface that generates the mechanical vibrations when actuated.

24. The handheld device according to claim 17, wherein the outer housing defines an opening at the skin facing surface, wherein the vibration unit is aligned with the opening.

25. The handheld device according to claim 24, further comprising a cover disposed in the opening and adjacent to the vibration unit.

26. The handheld device according to claim 17, wherein the vibration unit is aligned with the skin facing surface of the outer housing along an axis that is substantially perpendicular to the outer housing.

27. The handheld device according to claim 17, wherein the outer housing has a first end, a second end opposite the first end, a top housing component, a bottom housing component opposite the top housing component, and an opening defined by the bottom housing component, and the bottom housing component being configured to face the head of the user, wherein the vibration unit is aligned with the opening so that vibrations emanate from housing proximate the opening.

28. The handheld device according to claim 27, wherein the vibration unit is a tactile transducer configured to convert a current into mechanical vibrations.

29. The handheld device according to claim 27, wherein the vibration unit is a linear transducer.

30. The handheld device according to claim 27, wherein the vibration unit is a haptic element.

31. The handheld device according to claim 17, wherein the vibration unit is actuated responsive to detection of one or more predetermined brainwave patterns in the user.

32. A method of providing stimulation to a head of a user, the method comprising the steps of:
placing a skin facing surface of a handheld device proximate a head of a user;
powering the handheld device to activate a controller contained within the handheld device;
holding the handheld device in place proximate the head of the user so that a vibration unit in electronic communication with the controller generates vibrations that corresponds to the pattern sequence, thereby stimulating the user's head in accordance with the pattern sequence.

33. The method according to claim 32, wherein the pattern sequence is one of:
a plurality of pulses with each pulse having the same amplitude;
a plurality of pulses having a first pulse at a first amplitude and a second pulse at a second amplitude that is different from than the first amplitude; or
a plurality of pulses having a first pulse at a first amplitude and a second set of pulses with amplitudes, wherein the amplitudes in the second set of pulses are different from the first amplitude in the first pulse.

34. The method according to claim 32, wherein the pattern sequence is one of:
a plurality of pulses with each pulse having the same frequency;
a plurality of pulses having a first pulse at a first frequency and a second pulse at a second frequency that is different from than the first frequency;
a plurality of pulses that alternate in succession between a first pulse at a first frequency and a second set of pulses with frequencies, wherein the frequencies in the second set of pulses are different than the first frequency of the first pulse; or
a first set pulses and a second set of pulses, wherein the first set of pulses and the second set of pulses have a frequency that varies over a period of time.

35. The method according to claim 32, wherein the pattern sequence is one of:
a plurality of pulses with each pulse having the same pitch;
a plurality of pulses having a first pulse at a first pitch and a second pulse at a second pitch that is different from than the first pitch; or
a plurality of pulses having a first pulse at a first pitch and a second set of pulses with pitches, wherein the pitches of the second set of pulses are different than the first pitch.

36. The method according to claim 32, further comprising:
allowing access to a data file in memory of a controller, wherein the data file includes data that corresponds to a pattern sequence; and
in response to accessing the data file, generating a signal corresponding to the pattern sequence.

37. The method according to claim 32, wherein the vibration unit is a tactile transducer configured to convert a current into mechanical vibrations.

38. The method according to claim 32, wherein the vibration unit is a linear transducer.

39. The method according to claim 32, wherein the vibration unit is a haptic element.

40. The method according to claim 32, wherein the vibration unit is actuated responsive to detection of one or more predetermined brainwave patterns in the user.
